(19) United States
(12) Patent Application Publication
(10) Pub. No.: US 2024/0047070 A1
(43) Pub. Date: Feb. 8, 2024

(54) MACHINE LEARNING TECHNIQUES FOR GENERATING COHORTS AND PREDICTIVE MODELING BASED THEREOF

(71) Applicant: Optum, Inc., Minnetonka, MN (US)

(72) Inventors: Mark Gregory Megerian, Rochester, MN (US); Daniel George McCreary, St. Louis Park, MN (US)

(21) Appl. No.: 17/817,472

(22) Filed: Aug. 4, 2022

(51) Int. Cl. G16H 50/30

(52) U.S. Cl. CPC G16H 50/30

(57) ABSTRACT

The present disclosure provides methods, apparatus, systems, computing devices, and/or the like for performing risk prediction by receiving outer cohort definition data and inner cohort definition data, the outer cohort definition data representative of a target data domain with respect to a dataset, and the inner cohort definition data representative of a prediction feature with respect to the target data domain, determining one or more inner cohort features based at least in part on a knowledge graph data object using the inner cohort definition data, the knowledge graph data object including co-occurrence information of features from the dataset, and generating, using a predictive machine learning model, for each of one or more outer cohort entities associated with features in an outer cohort data subset, a risk score representative of a propensity of the outer cohort entity being an inner cohort entity associated with features in an inner cohort data subset.

### BACKGROUND

Various embodiments of the present disclosure address technical challenges related to performing predictive data analysis and provide solutions to address the efficiency and reliability shortcomings of existing predictive data analysis solutions.

### BRIEF SUMMARY

In general, various embodiments of the present disclosure provide methods, apparatus, systems, computing devices, computing entities, and/or the like for performing risk prediction with respect to data subsets.

In accordance with one aspect, a method is provided. In one embodiment, the method comprises: receiving, by a computing device, outer cohort definition data and inner cohort definition data, the outer cohort definition data representative of a target data domain with respect to a dataset, and the inner cohort definition data representative of a prediction feature with respect to the target data domain; determining, by the computing device, one or more inner cohort features based at least in part on a knowledge graph data object using the inner cohort definition data, the knowledge graph data object including co-occurrence information of features from the dataset; for each outer cohort entity of one or more outer cohort entities associated with features in an outer cohort data subset, generating, by the computing device and using a predictive machine learning model, a risk score representative of a propensity of the outer cohort entity being an inner cohort entity, wherein training the predictive machine learning model comprises: identifying an outer cohort data subset from the dataset based at least in part on the outer cohort definition data; for each entity associated with the outer cohort data subset, retrieving an input feature value set comprising input feature values associated with the outer cohort data entity that correspond to the one or more inner cohort features; for each entity associated with the outer cohort data subset, determining a membership indicator to the inner cohort data subset based at least in part on using the knowledge graph data object to determine correlation values between the input feature values associated with the outer cohort data subset and the one or more inner cohort features; generating training data based at least in part on: (i) each membership indicator, and (ii) each input feature value set; and training the predictive machine learning model based at least in part on the training data; and performing, by the computing device, one or more prediction-based actions based at least in part on the risk score.

In accordance with another aspect, an apparatus comprising at least one processor and at least one memory including computer program code is provided. In one embodiment, the at least one memory and the computer program code may be configured to, with the processor, cause the apparatus to: receive outer cohort definition data and inner cohort definition data, the outer cohort definition data representative of a target data domain with respect to a dataset, and the inner cohort definition data representative of a prediction feature with respect to the target data domain; determine one or more inner cohort features based at least in part on a knowledge graph data object using the inner cohort definition data, the knowledge graph data object including co-occurrence information of features from the dataset; for each outer cohort entity of one or more outer cohort entities associated with features in an outer cohort data subset, generating, by the computing device and using a predictive machine learning model, a risk score representative of a propensity of the outer cohort entity being an inner cohort entity, wherein training the predictive machine learning model comprises: identifying an outer cohort data subset from the dataset based at least in part on the outer cohort definition data; for each entity associated with the outer cohort data subset, retrieving an input feature value set comprising input feature values associated with the outer cohort data entity that correspond to the one or more inner cohort features; for each entity associated with the outer cohort data subset, determining a membership indicator to the inner cohort data subset based at least in part on using the knowledge graph data object to determine correlation values between the input feature values associated with the outer cohort data subset and the one or more inner cohort features; generating training data based at least in part on: (i) each membership indicator, and (ii) each input feature value set; and training the predictive machine learning model based at least in part on the training data; and perform one or more prediction-based actions based at least in part on the risk score.

In accordance with yet another aspect, a computer program product is provided. The computer program product may comprise at least one computer-readable storage medium having computer-readable program code portions stored therein, the computer-readable program code portions comprising executable portions configured to: receive outer cohort definition data and inner cohort definition data, the outer cohort definition data representative of a target data domain with respect to a dataset, and the inner cohort definition data representative of a prediction feature with respect to the target data domain; determine one or more inner cohort features based at least in part on a knowledge graph data object using the inner cohort definition data, the knowledge graph data object including co-occurrence information of features from the dataset; for each outer cohort entity of one or more outer cohort entities associated with features in an outer cohort data subset, generating, by the computing device and using a predictive machine learning model, a risk score representative of a propensity of the outer cohort entity being an inner cohort entity, wherein training the predictive machine learning model comprises: identifying an outer cohort data subset from the dataset based at least in part on the outer cohort definition data; for each entity associated with the outer cohort data subset, retrieving an input feature value set comprising input feature values associated with the outer cohort data entity that correspond to the one or more inner cohort features; for each entity associated with the outer cohort data subset, determining a membership indicator to the inner cohort data subset based at least in part on using the knowledge graph data object to determine correlation values between the input feature values associated with the outer cohort data subset and the one or more inner cohort features; generating training data based at least in part on: (i) each membership indicator, and (ii) each input feature value set; and training the predictive machine learning model based at least in part on the training data; and perform one or more prediction-based actions based at least in part on the risk score.

### BRIEF DESCRIPTION OF THE DRAWINGS

Having thus described the disclosure in general terms, reference will now be made to the accompanying drawings, which are not necessarily drawn to scale, and wherein:

FIG. 1 provides an exemplary overview of an architecture that can be used to practice embodiments of the present disclosure.

FIG. 2 provides an example predictive data analysis computing entity in accordance with some embodiments discussed herein.

FIG. 3 provides an example client computing entity in accordance with some embodiments discussed herein.

FIG. 4 is a flowchart diagram of an example process for training a predictive machine learning model in accordance with some embodiments discussed herein.

FIG. 5 illustrates an exemplary knowledge graph in accordance with some embodiments discussed herein.

FIG. 6 illustrates exemplary inner cohort definition data in accordance with some embodiments discussed herein.

FIG. 7 is a flowchart diagram of an example process for performing predictive operations on a target data domain in accordance with some embodiments discussed herein.

FIG. 8 provides an operational example of a set of inputs for generating predictive events corresponding to at risk patients in accordance with some embodiments discussed herein.

### DETAILED DESCRIPTION

Various embodiments of the present disclosure now will be described more fully hereinafter with reference to the accompanying drawings, in which some, but not all, embodiments of the disclosures are shown. Indeed, these disclosures may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “exemplary” are used to be examples with no indication of quality level. Like numbers refer to like elements throughout. Moreover, while certain embodiments of the present disclosure are described with reference to predictive data analysis, one of ordinary skill in the art will recognize that the disclosed concepts can be used to perform other types of data analysis.

### I. Overview and Technical Improvements

The present disclosure provides a prediction system that allows users to describe a target data domain for analysis (e.g., population of members) based upon the presence of features in data within a dataset (e.g., procedure or diagnosis codes in their medical history), and automatically predicts whether entities associated with the target data domain are at risk of prediction features (e.g., developing a set of diagnosis or procedure codes) defined by the user. The disclosed prediction system may also use data from a connected knowledge graph data object to discover correlated features, and in this manner, perform automated feature selection. Accordingly, prediction modeling and risk stratification can be performed by non-programmers, simply by providing a definition of a feature being predicted. The examples described herewith utilize diagnosed conditions as a feature, but the disclosed prediction system can be equally applied to additional features such as medications, past procedures, or lab results.

Various embodiments of the present disclosure make important technical contributions to improving predictive accuracy of predictive machine learning models by identifying targeted cohort data subsets, which in turn improves training speed and training efficiency of training predictive machine learning models. It is well-understood in the relevant art that there is typically a tradeoff between predictive accuracy and training speed, such that it is trivial to improve training speed by reducing predictive accuracy, and thus the real challenge is to improve training speed without sacrificing predictive accuracy through innovative model architectures, see, e.g., Sun et al., Feature-Frequency-Adaptive On-line Training for Fast and Accurate Natural Language Processing in 40(3) Computational Linguistic 563 at Abst. (“Typically, we need to make a tradeoff between speed and accuracy. It is trivial to improve the training speed via sacrificing accuracy or to improve the accuracy via sacrificing speed. Nevertheless, it is nontrivial to improve the training speed and the accuracy at the same time”). Accordingly, techniques that improve predictive accuracy without harming training speed, such as the techniques described herein, enable improving training speed given a constant predictive accuracy. In doing so, the techniques described herein improving efficiency and speed of training predictive machine learning models, thus reducing the number of computational operations needed and/or the amount of training data entries needed to train predictive machine learning models. Accordingly, the techniques described herein improve at least one of the computational efficiency, storage-wise efficiency, and speed of training machine learning models.

For example, various embodiments of the present disclosure improve predictive accuracy of predictive machine learning models by identifying targeted cohort data subsets. As described herein, feature engineering and data cleaning processes used in performing data analysis and building machine learning models are computationally intensive. In particular, certain kinds of data, such as healthcare data is particularly complex and extensive than many other disciplines. For this reason, it is important to have techniques available to combine the need for rapid analysis and discovery. Furthermore, techniques that analyze massive amounts of data in order to produce a “black box” model are not useful, for example, in a healthcare setting where there is a need to understand “why” a member is at high risk for a condition.

However, in accordance with various embodiments of the present disclosure, a predictive machine learning model may be trained to predict whether data within an outer cohort data subset that is not a member of an inner cohort data subset will become a member of the inner cohort data subset. The outer cohort data subset may comprise a portion of a dataset representative of a target data domain and the inner cohort data subset may comprise a portion of the dataset representative of a feature for prediction on the target data domain. Accordingly, initializing the predictive machine learning model may require minimal input, such as description of criteria for the outer cohort data subset and the inner cohort data subset. This technique will lead to higher accuracy of performing predictive operations as needed on certain sets of data. In doing so, the techniques described herein improve efficiency and speed of training predictive machine learning models, thus reducing the number of computational operations needed and/or the amount of training data entries needed to train predictive machine learning models. Accordingly, the techniques described herein improve at least one of the computational efficiency, storage-wise efficiency, and speed of training predictive machine learning models.

Various embodiments of the present invention enable techniques for using feature data associated with one predictive task, such as a predictive task related to an inner cohort membership prediction, to train a predictive machine learning model that is configured to perform predictive inferences related to an outer cohort membership prediction. In this way, the noted embodiments of the present invention enable training a predictive machine learning model that is configured to perform predictive inferences related to an outer cohort membership prediction without having to store training data whose feature set is specifically generated for the outer cohort membership prediction task, which in turn reduces storage requirements for training the predictive machine learning model and thus enhances storage-wise efficiency of training the predictive machine learning model. Accordingly, various embodiments of the present invention improve storage-wise efficiency of training predictive machine learning models that are configured to perform outer cohort membership prediction tasks.

### II. Definitions

The term “outer cohort definition data” may refer to a data construct that describes a subset of data from a dataset for defining an outer cohort data subset representative of a target data domain. The outer cohort definition data may include criteria, such as one or more features characterizing data from a dataset for inclusion in an outer cohort data subset. As an example, the one or more features may include procedure or diagnosis codes, and the data from the dataset may comprise medical history data. In another example, the one or more features may include age, gender, or demographics. The outer cohort definition data can be used to define, for example, a population as a target data domain to create an outer cohort data subset, such as people in wheelchairs, based at least in part on whether data in the dataset includes the one or more features.

The term “outer cohort data subset” may refer to a data construct that describes a portion of a dataset associated with a target data domain (e.g., a population for analysis). The outer cohort data subset may be defined based at least in part on outer cohort definition data. According to various embodiments of the present disclosure, an outer cohort data subset may be generated by identifying data from a dataset including criteria based at least in part on outer cohort definition data.

The term “outer cohort entity” may refer to a data construct that describes an entity associated with one or more features belonging to an outer cohort data subset. As an example, an outer cohort entity may comprise a patient corresponding to medical history data that is in an outer cohort data subset.

The term “inner cohort definition data” may refer to a data construct that describes a further subset of a subset of data (e.g., an outer cohort data subset representative of a target data domain) from a dataset for defining an inner cohort data subset. The inner cohort definition data may include criteria, such as one or more prediction features with respect to an outer cohort data subset. As an example, the one or more prediction features may include a target prediction of procedures or diagnoses regarding data (e.g., medical history data) associated with an outer cohort data subset. The inner cohort definition data can be used to define, for example, a subpopulation with respect to a population (e.g., defined by outer cohort definition data), such as a portion of people in wheelchairs that will develop a pressure ulcer.

The term “inner cohort data subset” may refer to a data construct that describes a portion of a dataset including a feature for prediction within an outer cohort data subset (representative of a target data domain). The inner cohort data subset may be defined based at least in part on inner cohort definition data. According to various embodiments of the present disclosure, an inner cohort data subset may be generated by identifying data from an outer cohort data subset including features defined by inner cohort definition data.

The term “inner cohort entity” may refer to a data construct that describes an entity associated with one or more features belonging to an inner cohort data subset. As an example, an inner cohort entity may comprise a patient corresponding to medical history data that is in an inner cohort data subset.

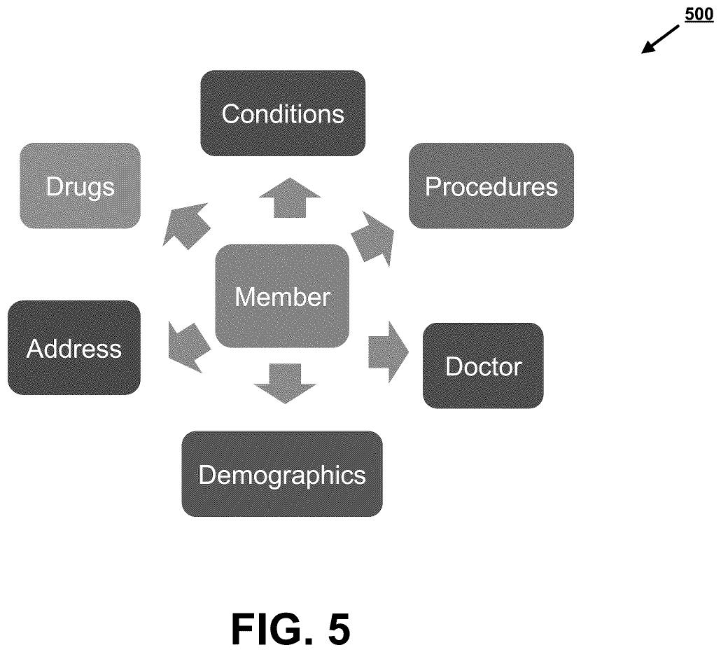

The term “knowledge graph” may refer to a data construct that describes a graph-structured data model that stores interlinked descriptions of entities. A knowledge graph may include a network of nodes, where each node is representative of one or more features associated with an entity from a dataset, and edges representative of relationships (e.g., co-occurrence) between the features. For example, a knowledge graph may comprise associations of correlated medical conditions, diagnosis codes, medications, demographics, or procedure codes based at least in part on medical history data of patients. FIG. 5 provides an exemplary depiction of a knowledge graph data object 500 in accordance with some embodiments of the present disclosure. In one embodiment, a knowledge graph may be created from a dataset using a semantic embedding of each data item from the dataset to semantically connect items based at least in part on correlation values between items.

The term “correlation values” may refer to a data construct that describes a statistical measure of a strength of a relationship between two features. In one embodiment, correlation values may be representative of co-occurrence frequency between a plurality of features, wherein each correlation value comprises a co-occurrence frequency measure for a feature pair. In one embodiment, correlation values may comprise an odds ratio. For example, the odds ratio may comprise a statistic that quantifies strength of association between two features defined as a ratio of the odds of a first feature in the presence of a second feature and the odds of the first feature in the absence of the second feature, or a ratio of the odds of the second feature in the presence of the first feature and the odds of the second feature in the absence of the first feature. Two features may be independent of each other if, for example, the odds of one feature are the same in either the presence or absence of the other feature. Features may be positively correlated or negatively correlated. Positive correlation may comprise, compared to an absence of one feature, the present of the one feature raises the odds of the other feature. Conversely, negative correlation may comprise, compared to an absence of one feature, the presence of the one feature reduces the odds of the other feature.

The term “co-occurrence frequency” may refer to a data construct that describes a strength of relationship between two features. In some embodiments, the relationship may comprise a semantic proximity of the two features. For example, co-occurrence frequency may comprise an above-chance frequency of two given features coinciding or existing within a structure of text.

The term “co-occurrence frequency measure” may refer to a data construct that describes a value corresponding to a measurement of co-occurrence frequency. The measurement of co-occurrence frequency may comprise correlation values that are calculated for a feature pair.

The term “predictive machine learning model” may refer to a data construct that describes parameters, hyperparameters, and/or defined operations of a machine learning model that is configured to generate a risk score representative of a propensity of an outer cohort entity with respect to an inner cohort entity. As described above, the outer cohort entity may comprise an entity associated with features in an outer cohort data subset and the inner cohort entity may comprise an entity associated with features in an inner cohort data subset. According to various embodiments of the present disclosure, the predictive machine learning model may generate the risk score for data corresponding to entities within an outer cohort data subset based at least in part on an inner cohort definition. The risk score may be used by a computing device to perform one or more prediction-based actions. The predictive machine learning model may be applied to any prediction task that are concentrated on certain populations. Some examples, in the healthcare space, include but are not limited to, predicting future health outcomes using medical data and identifying members that are at higher risk of developing a diagnosis. In an example, the predictive machine learning model may predict medical diagnosis associated with members of a certain healthcare demographics and recommend procedures or treatment to a medical decision aid system.

The term “risk score” may refer to a data construct that describes an output of a predictive machine learning model. A risk score may comprise a probability of an entity associated with features in an outer cohort data subset will become an entity associated with features in an inner cohort data subset. For example, the risk score may define a probability of wheelchair-bound patients (outer cohort data subset) that develop a pressure ulcer (inner cohort data subset). Other examples may include predicting cancer patients that develop adverse reactions to chemotherapy, and diabetics that develop foot problems.

### III. Computer Program Products, Methods, and Computing Entities

Embodiments of the present disclosure may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present disclosure may also be implemented as methods, apparatus, systems, computing devices, computing entities, and/or the like. As such, embodiments of the present disclosure may take the form of an apparatus, system, computing device, computing entity, and/or the like executing instructions stored on a computer-readable storage medium to perform certain steps or operations. Thus, embodiments of the present disclosure may also take the form of an entirely hardware embodiment, an entirely computer program product embodiment, and/or an embodiment that comprises combination of computer program products and hardware performing certain steps or operations.

Embodiments of the present disclosure are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some exemplary embodiments, retrieval, loading, and/or execution may be performed in parallel such that multiple instructions are retrieved, loaded, and/or executed together. Thus, such embodiments can produce specifically-configured machines performing the steps or operations specified in the block diagrams and flowchart illustrations. Accordingly, the block diagrams and flowchart illustrations support various combinations of embodiments for performing the specified instructions, operations, or steps.

### IV. Exemplary System Architecture

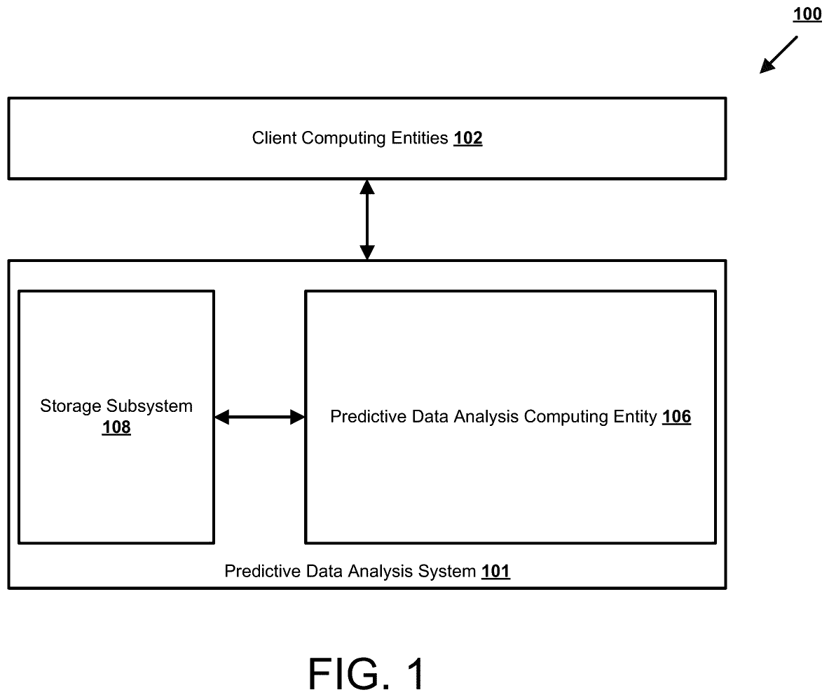

FIG. 1 is a schematic diagram of an example architecture 100 for performing predictive data analysis. The architecture 100 includes a predictive data analysis system 101 configured to receive predictive data analysis requests from client computing entities 102, process the predictive data analysis requests to generate predictions, provide the generated predictions to the client computing entities 102, and automatically perform prediction-based actions based at least in part on the generated predictions.

An example of a prediction-based action that can be performed using the predictive data analysis system 101 is a request for generating a diagnosis code for a clinical text. For example, in accordance with various embodiments of the present disclosure, a predictive machine learning model may be trained to predict whether data within an outer cohort data subset that is not a member of an inner cohort data subset will become a member of the inner cohort data subset. The outer cohort data subset may comprise a portion of a dataset representative of a target data domain and the inner cohort data subset may comprise a portion of the dataset representative of a feature for prediction on the target data domain. Accordingly, initializing the predictive machine learning model may require minimal input, such as description of criteria for the outer cohort data subset and the inner cohort data subset. This technique will lead to higher accuracy of performing predictive operations as needed on certain sets of data. In doing so, the techniques described herein improve efficiency and speed of training predictive machine learning models, thus reducing the number of computational operations needed and/or the amount of training data entries needed to train predictive machine learning models. Accordingly, the techniques described herein improve at least one of the computational efficiency, storage-wise efficiency, and speed of training predictive machine learning models.

In some embodiments, predictive data analysis system 101 may communicate with at least one of the client computing entities 102 using one or more communication networks. Examples of communication networks include any wired or wireless communication network including, for example, a wired or wireless local area network (LAN), personal area network (PAN), metropolitan area network (MAN), wide area network (WAN), or the like, as well as any hardware, software and/or firmware required to implement it (such as, e.g., network routers, and/or the like).

The predictive data analysis system 101 may include a predictive data analysis computing entity 106 and a storage subsystem 108. The predictive data analysis computing entity 106 may be configured to receive predictive data analysis requests from one or more client computing entities 102, process the predictive data analysis requests to generate predictions corresponding to the predictive data analysis requests, provide the generated predictions to the client computing entities 102, and automatically perform prediction-based actions based at least in part on the generated predictions.

The storage subsystem 108 may be configured to store input data used by the predictive data analysis computing entity 106 to perform predictive data analysis as well as model definition data used by the predictive data analysis computing entity 106 to perform various predictive data analysis tasks. The storage subsystem 108 may include one or more storage units, such as multiple distributed storage units that are connected through a computer network. Each storage unit in the storage subsystem 108 may store at least one of one or more data assets and/or one or more data about the computed properties of one or more data assets. Moreover, each storage unit in the storage subsystem 108 may include one or more non-volatile storage or memory media including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

A. Exemplary Predictive Data Analysis Computing Entity

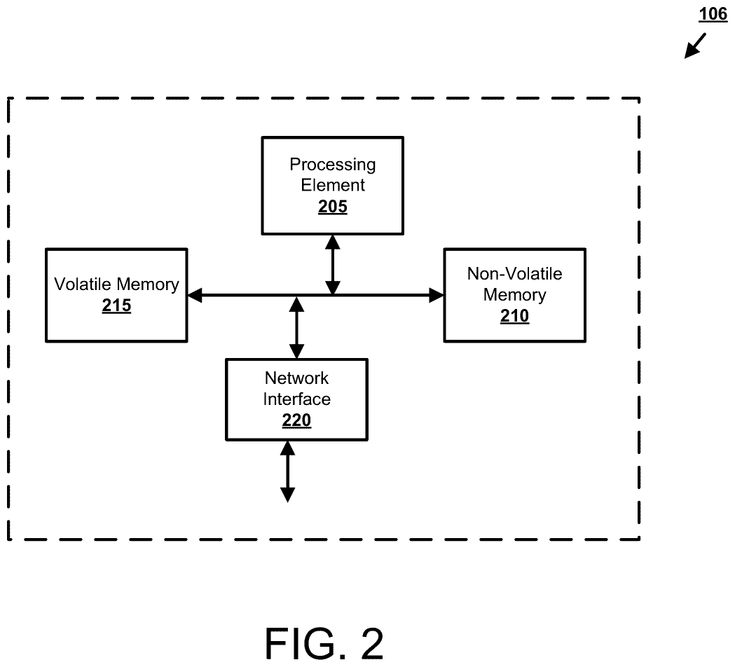

FIG. 2 provides a schematic of a predictive data analysis computing entity 106 according to one embodiment of the present disclosure. In general, the terms computing entity, computer, entity, device, system, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Such functions, operations, and/or processes may include, for example, transmitting, receiving, operating on, processing, displaying, storing, determining, creating/generating, monitoring, evaluating, comparing, and/or similar terms used herein interchangeably. In one embodiment, these functions, operations, and/or processes can be performed on data, content, information, and/or similar terms used herein interchangeably.

As indicated, in one embodiment, the predictive data analysis computing entity 106 may also include one or more communications interfaces 220 for communicating with various computing entities, such as by communicating data, content, information, and/or similar terms used herein interchangeably that can be transmitted, received, operated on, processed, displayed, stored, and/or the like.

As shown in FIG. 2, in one embodiment, the predictive data analysis computing entity 106 may include, or be in communication with, one or more processing elements 205 (also referred to as processors, processing circuitry, and/or similar terms used herein interchangeably) that communicate with other elements within the predictive data analysis computing entity 106 via a bus, for example. As will be understood, the processing element 205 may be embodied in a number of different ways.

For example, the processing element 205 may be embodied as one or more complex programmable logic devices (CPLDs), microprocessors, multi-core processors, coprocessing entities, application-specific instruction-set processors (ASIPs), microcontrollers, and/or controllers. Further, the processing element 205 may be embodied as one or more other processing devices or circuitry. The term circuitry may refer to an entirely hardware embodiment or a combination of hardware and computer program products. Thus, the processing element 205 may be embodied as integrated circuits, application specific integrated circuits (ASICs), field programmable gate arrays (FPGAs), programmable logic arrays (PLAs), hardware accelerators, other circuitry, and/or the like.

As will therefore be understood, the processing element 205 may be configured for a particular use or configured to execute instructions stored in volatile or non-volatile media or otherwise accessible to the processing element 205. As such, whether configured by hardware or computer program products, or by a combination thereof, the processing element 205 may be capable of performing steps or operations according to embodiments of the present disclosure when configured accordingly.

In one embodiment, the predictive data analysis computing entity 106 may further include, or be in communication with, non-volatile media (also referred to as non-volatile storage, memory, memory storage, memory circuitry and/or similar terms used herein interchangeably). In one embodiment, the non-volatile storage or memory may include one or more non-volatile storage or memory media 210, including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

As will be recognized, the non-volatile storage or memory media may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity-relationship model, object model, document model, semantic model, graph model, and/or the like.

In one embodiment, the predictive data analysis computing entity 106 may further include, or be in communication with, volatile media (also referred to as volatile storage, memory, memory storage, memory circuitry and/or similar terms used herein interchangeably). In one embodiment, the volatile storage or memory may also include one or more volatile storage or memory media 215, including, but not limited to, RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like.

As will be recognized, the volatile storage or memory media may be used to store at least portions of the databases, database instances, database management systems, data, applications, programs, program modules, scripts, source code, object code, byte code, compiled code, interpreted code, machine code, executable instructions, and/or the like being executed by, for example, the processing element 205. Thus, the databases, database instances, database management systems, data, applications, programs, program modules, scripts, source code, object code, byte code, compiled code, interpreted code, machine code, executable instructions, and/or the like may be used to control certain aspects of the operation of the predictive data analysis computing entity 106 with the assistance of the processing element 205 and operating system.

As indicated, in one embodiment, the predictive data analysis computing entity 106 may also include one or more communications interfaces 220 for communicating with various computing entities, such as by communicating data, content, information, and/or similar terms used herein interchangeably that can be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the predictive data analysis computing entity 106 may be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

Although not shown, the predictive data analysis computing entity 106 may include, or be in communication with, one or more input elements, such as a keyboard input, a mouse input, a touch screen/display input, motion input, movement input, audio input, pointing device input, joystick input, keypad input, and/or the like. The predictive data analysis computing entity 106 may also include, or be in communication with, one or more output elements (not shown), such as audio output, video output, screen/display output, motion output, movement output, and/or the like.

B. Exemplary Client Computing Entity

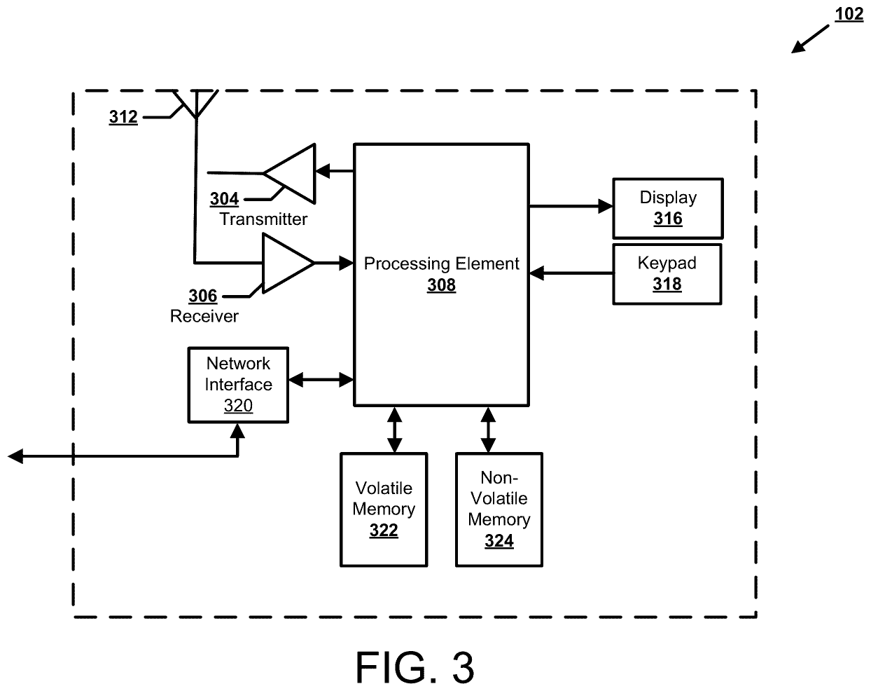

FIG. 3 provides an illustrative schematic representative of a client computing entity 102 that can be used in conjunction with embodiments of the present disclosure. In general, the terms device, system, computing entity, entity, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Client computing entities 102 can be operated by various parties. As shown in FIG. 3, the client computing entity 102 can include an antenna 312, a transmitter 304 (e.g., radio), a receiver 306 (e.g., radio), and a processing element 308 (e.g., CPLDs, microprocessors, multi-core processors, coprocessing entities, ASIPs, microcontrollers, and/or controllers) that provides signals to and receives signals from the transmitter 304 and receiver 306, correspondingly.

The signals provided to and received from the transmitter 304 and the receiver 306, correspondingly, may include signaling information/data in accordance with air interface standards of applicable wireless systems. In this regard, the client computing entity 102 may be capable of operating with one or more air interface standards, communication protocols, modulation types, and access types. More particularly, the client computing entity 102 may operate in accordance with any of a number of wireless communication standards and protocols, such as those described above with regard to the predictive data analysis computing entity 106. In a particular embodiment, the client computing entity 102 may operate in accordance with multiple wireless communication standards and protocols, such as UMTS, CDMA2000, 1×RTT, WCDMA, GSM, EDGE, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the client computing entity 102 may operate in accordance with multiple wired communication standards and protocols, such as those described above with regard to the predictive data analysis computing entity 106 via a network interface 320.

Via these communication standards and protocols, the client computing entity 102 can communicate with various other entities using concepts such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM dialer). The client computing entity 102 can also download changes, add-ons, and updates, for instance, to its firmware, software (e.g., including executable instructions, applications, program modules), and operating system.

According to one embodiment, the client computing entity 102 may include location determining aspects, devices, modules, functionalities, and/or similar words used herein interchangeably. For example, the client computing entity 102 may include outdoor positioning aspects, such as a location module adapted to acquire, for example, latitude, longitude, altitude, geocode, course, direction, heading, speed, universal time (UTC), date, and/or various other information/data. In one embodiment, the location module can acquire data, sometimes known as ephemeris data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This data can be collected using a variety of coordinate systems, such as the Decimal Degrees (DD); Degrees, Minutes, Seconds (DMS); Universal Transverse Mercator (UTM); Universal Polar Stereographic (UPS) coordinate systems; and/or the like. Alternatively, the location information/data can be determined by triangulating the client computing entity's 102 position in connection with a variety of other systems, including cellular towers, Wi-Fi access points, and/or the like. Similarly, the client computing entity 102 may include indoor positioning aspects, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices (e.g., smartphones, laptops) and/or the like. For instance, such technologies may include the iBeacons, Gimbal proximity beacons, Bluetooth Low Energy (BLE) transmitters, NFC transmitters, and/or the like. These indoor positioning aspects can be used in a variety of settings to determine the location of someone or something to within inches or centimeters.

The client computing entity 102 may also comprise a user interface (that can include a display 316 coupled to a processing element 308) and/or a user input interface (coupled to a processing element 308). For example, the user interface may be a user application, browser, user interface, and/or similar words used herein interchangeably executing on and/or accessible via the client computing entity 102 to interact with and/or cause display of information/data from the predictive data analysis computing entity 106, as described herein. The user input interface can comprise any of a number of devices or interfaces allowing the client computing entity 102 to receive data, such as a keypad 318 (hard or soft), a touch display, voice/speech or motion interfaces, or other input device. In embodiments including a keypad 318, the keypad 318 can include (or cause display of) the conventional numeric (0-9) and related keys (#, *), and other keys used for operating the client computing entity 102 and may include a full set of alphabetic keys or set of keys that may be activated to provide a full set of alphanumeric keys. In addition to providing input, the user input interface can be used, for example, to activate or deactivate certain functions, such as screen savers and/or sleep modes.

The client computing entity 102 can also include volatile storage or memory 322 and/or non-volatile storage or memory 324, which can be embedded and/or may be removable. For example, the non-volatile memory may be ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. The volatile and non-volatile storage or memory can store databases, database instances, database management systems, data, applications, programs, program modules, scripts, source code, object code, byte code, compiled code, interpreted code, machine code, executable instructions, and/or the like to implement the functions of the client computing entity 102. As indicated, this may include a user application that is resident on the entity or accessible through a browser or other user interface for communicating with the predictive data analysis computing entity 106 and/or various other computing entities.

In another embodiment, the client computing entity 102 may include one or more components or functionality that are the same or similar to those of the predictive data analysis computing entity 106, as described in greater detail above. As will be recognized, these architectures and descriptions are provided for exemplary purposes only and are not limiting to the various embodiments.

In various embodiments, the client computing entity 102 may be embodied as an artificial intelligence (AI) computing entity, such as an Amazon Echo, Amazon Echo Dot, Amazon Show, Google Home, and/or the like. Accordingly, the client computing entity 102 may be configured to provide and/or receive information/data from a user via an input/output mechanism, such as a display, a camera, a speaker, a voice-activated input, and/or the like. In certain embodiments, an AI computing entity may comprise one or more predefined and executable program algorithms stored within an onboard memory storage module, and/or accessible over a network. In various embodiments, the AI computing entity may be configured to retrieve and/or execute one or more of the predefined program algorithms upon the occurrence of a predefined trigger event.

### V. Exemplary System Operations

As described below, various embodiments of the present disclosure make important technical contributions to improving predictive accuracy of predictive machine learning models by identifying targeted cohort data subsets, which in turn improves training speed and training efficiency of training predictive machine learning models. It is well-understood in the relevant art that there is typically a tradeoff between predictive accuracy and training speed, such that it is trivial to improve training speed by reducing predictive accuracy, and thus the real challenge is to improve training speed without sacrificing predictive accuracy through innovative model architectures, see, e.g., Sun et al., Feature-Frequency-Adaptive On-line Training for Fast and Accurate Natural Language Processing in 40(3) Computational Linguistic 563 at Abst. (“Typically, we need to make a tradeoff between speed and accuracy. It is trivial to improve the training speed via sacrificing accuracy or to improve the accuracy via sacrificing speed. Nevertheless, it is nontrivial to improve the training speed and the accuracy at the same time”). Accordingly, techniques that improve predictive accuracy without harming training speed, such as the techniques described herein, enable improving training speed given a constant predictive accuracy. In doing so, the techniques described herein improving efficiency and speed of training predictive machine learning models, thus reducing the number of computational operations needed and/or the amount of training data entries needed to train predictive machine learning models. Accordingly, the techniques described herein improve at least one of the computational efficiency, storage-wise efficiency, and speed of training machine learning models.

Moreover, as further described below, various embodiments of the present invention enable techniques for using feature data associated with one predictive task, such as a predictive task related to an inner cohort membership prediction, to train a predictive machine learning model that is configured to perform predictive inferences related to an outer cohort membership prediction. In this way, the noted embodiments of the present invention enable training a predictive machine learning model that is configured to perform predictive inferences related to an outer cohort membership prediction without having to store training data whose feature set is specifically generated for the outer cohort membership prediction task, which in turn reduces storage requirements for training the predictive machine learning model and thus enhances storage-wise efficiency of training the predictive machine learning model. Accordingly, various embodiments of the present invention improve storage-wise efficiency of training predictive machine learning models that are configured to perform outer cohort membership prediction tasks.

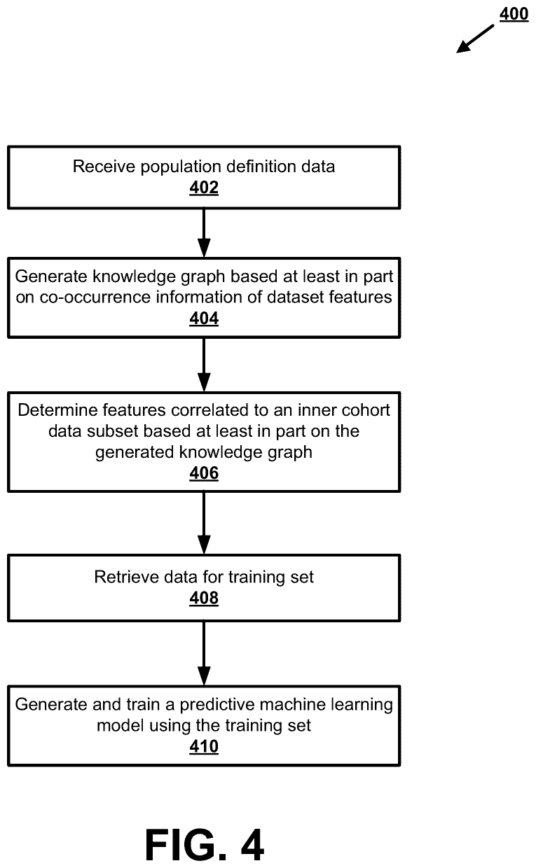

FIG. 4 is a flowchart diagram of an example process 400 for training a predictive machine learning model according to some embodiments disclosed herewith. Via the various steps/operations of the process 400, the predictive data analysis computing entity 106 can train a predictive machine learning model to generate risk scores for predicting outcomes for a target data domain.

The process 400 begins at step/operation 402 when the predictive data analysis computing entity 106 receives population definition data. The population definition data may comprise outer cohort definition data and inner cohort definition data. The outer cohort definition data may include criteria, such as features, for defining an outer cohort data subset comprising a subset of data from a dataset representative of a target data domain with respect to a dataset. As an example, the outer cohort definition data may be used to define a population from a dataset of medical data. The outer cohort definition data may include features associated with procedure or diagnosis codes for defining the population. The outer cohort definition data may also include criteria for age, gender, demographics, or any other detail known to one of ordinary skill in the art to define the population.

The inner cohort definition data may include criteria, such as feature, for defining an inner cohort data subset comprising a further subset of data from a defined subset of data representative of a prediction feature with respect to a target data domain according to outer cohort definition data. As an example, the inner cohort definition data may be used to define certain members from a defined population according to outer cohort definition data. The inner cohort definition data may include features associated with procedure or diagnosis codes for prediction from the defined population.

According to various embodiments of the present disclosure, the outer cohort definition data and the inner cohort definition data may be used to create a training set for a predictive machine learning model to discover risk factors of a specified population to develop a specific condition, which is discussed in further detail below. Examples may include determining risk of wheelchair-bound patients developing a pressure ulcer, cancer patients developing adverse reactions to chemotherapy, and diabetics developing foot problems.

However, as described herein, in accordance with various embodiments of the present disclosure, a predictive machine learning model may be trained to predict whether data within an outer cohort data subset that is not a member of an inner cohort data subset will become a member of the inner cohort data subset. The outer cohort data subset may comprise a portion of a dataset representative of a target data domain and the inner cohort data subset may comprise a portion of the dataset representative of a feature for prediction on the target data domain. Accordingly, initializing the predictive machine learning model may require minimal input, such as description of criteria for the outer cohort data subset and the inner cohort data subset. This technique will lead to higher accuracy of performing predictive operations as needed on certain sets of data. In doing so, the techniques described herein improve efficiency and speed of training predictive machine learning models, thus reducing the number of computational operations needed and/or the amount of training data entries needed to train predictive machine learning models. Accordingly, the techniques described herein improve at least one of the computational efficiency, storage-wise efficiency, and speed of training predictive machine learning models.

At step/operation 404, the predictive data analysis computing entity 106 generates a knowledge graph data object based at least in part on co-occurrence information of dataset features. Accordingly, in some embodiments, via performing step/operation 404, the predictive data analysis computing entity 106 retrieves and identify features from a dataset. The dataset may comprise a collection of data from one or more sources that may be queried from, such as databases, and joined. As an example, features, such as diagnosis codes, medications, demographics, or procedure codes may be retrieved to create a knowledge graph data object.

In some embodiments, a knowledge graph data object describes a graph-structured data model that stores interlinked descriptions of entities. A knowledge graph data object may include a network of nodes, where each node is representative of one or more features associated with an entity from a dataset, and edges representative of relationships (e.g., co-occurrence) between the features. For example, a knowledge graph data object may comprise associations of correlated medical conditions, diagnosis codes, medications, demographics, or procedure codes based at least in part on medical history data of patients. Referring to FIG. 5, an exemplary depiction of a knowledge graph data object 500 is provided in accordance with some embodiments of the present disclosure.

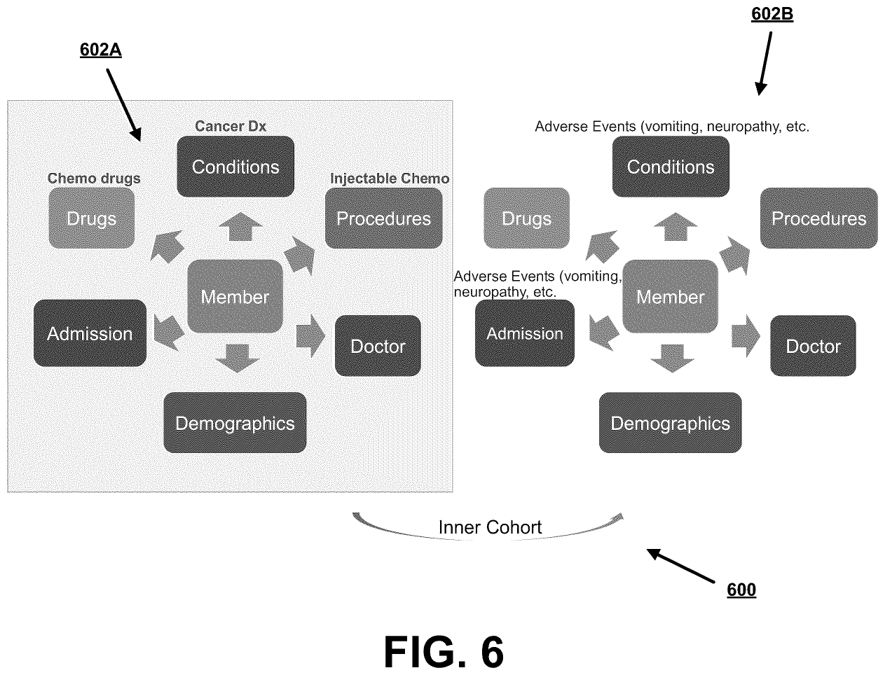

Referring back to FIG. 4, at step/operation 406, the predictive data analysis computing entity 106 determines features correlated to an inner cohort data subset based at least in part on the generated knowledge graph data object. Correlation values may be calculated between features from the dataset based at least in part on relationships found in the knowledge graph data object. As an example, referring to FIG. 6, an exemplary determination process 600 of adverse events (features correlated to an inner cohort data subset) based on a knowledge graph data object 602 is depicted.

In some embodiments, correlation values describe a statistical measure of a strength of a relationship between two features. In one embodiment, correlation values may be representative of co-occurrence frequency between a plurality of features, wherein each correlation value comprises a co-occurrence frequency measure for a feature pair. In one embodiment, correlation values may comprise an odds ratio. For example, the odds ratio may comprise a statistic that quantifies strength of association between two features defined as a ratio of the odds of a first feature in the presence of a second feature and the odds of the first feature in the absence of the second feature, or a ratio of the odds of the second feature in the presence of the first feature and the odds of the second feature in the absence of the first feature. Two features may be independent of each other if, for example, the odds of one feature are the same in either the presence or absence of the other feature. Features may be positively correlated or negatively correlated. Positive correlation may comprise, compared to an absence of one feature, the present of the one feature raises the odds of the other feature. Conversely, negative correlation may comprise, compared to an absence of one feature, the presence of the one feature reduces the odds of the other feature.

In some embodiments, co-occurrence frequency describes a strength of relationship between two features. In some embodiments, the relationship may comprise a semantic proximity of the two features. For example, co-occurrence frequency may comprise an above-chance frequency of two given features coinciding or existing within a structure of text.

In some embodiments, co-occurrence frequency measure describes a value corresponding to a measurement of co-occurrence frequency. The measurement of co-occurrence frequency may comprise correlation values that are calculated for a feature pair.

Referring back to FIG. 4, at step/operation 408, the predictive data analysis computing entity 106 retrieves data for a training set. Retrieving training set data may comprise retrieving features of outer cohort entities from the dataset. In some embodiments, an outer cohort entity describes an entity associated with one or more features belonging to an outer cohort data subset. An affirmative member indicator may be assigned to each entity associated with the outer cohort data subset. Furthermore, training data for each entity associated with the outer cohort data subset that is associated with an affirmative membership indicator, may include a training data entry that comprises an input feature value set for the entity and a ground-truth output that is generated based at least in part on the affirmative membership indicator. As an example, an outer cohort entity may comprise a patient corresponding to medical history data that is in an outer cohort data subset. In some embodiments, an outer cohort data subset describes a portion of a dataset associated with a target data domain (e.g., a population for analysis). The outer cohort data subset may be defined based at least in part on outer cohort definition data. According to various embodiments of the present disclosure, an outer cohort data subset may be generated by identifying data from a dataset including criteria based at least in part on outer cohort definition data.

For each outer cohort entity, the predictive data analysis computing entity 106 via performing step/operation 408 also determines whether the outer cohort entity is also an inner cohort entity by determining membership to the inner cohort data subset based at least in part on the determined features correlated to the inner cohort data subset.

In some embodiments, an “inner cohort entity” describes an entity associated with one or more features belonging to an inner cohort data subset. As an example, an inner cohort entity may comprise a patient corresponding to medical history data that is in an inner cohort data subset.

In some embodiments, an inner cohort data subset describes a portion of a dataset including a feature for prediction within an outer cohort data subset (representative of a target data domain). The inner cohort data subset may be defined based at least in part on inner cohort definition data. According to various embodiments of the present disclosure, an inner cohort data subset may be generated by identifying data from an outer cohort data subset including features defined by inner cohort definition data.

At step/operation 410, the predictive data analysis computing entity 106 generates and trains a predictive machine learning model using the training set. In some embodiments, a predictive machine learning model describes parameters, hyperparameters, and/or defined operations of a machine learning model that is configured to generate a risk score representative of a propensity of an outer cohort entity with respect to an inner cohort entity. As described above, the outer cohort entity may comprise an entity associated with features in an outer cohort data subset and the inner cohort entity may comprise an entity associated with features in an inner cohort data subset. The predictive machine learning model may be based on at least one of neural network, random forest, logistic regression, and gradient boosting learning techniques.

According to various embodiments of the present disclosure, the step/operation 402 through step/operation 410 may be repeated to train one or more predictive machine learning models to generate risk scores for predicting outcomes for a plurality of permutations of the population definition data (e.g., for a variety of outer cohort and inner cohort combinations).

According to various embodiments of the present disclosure, the predictive machine learning model may generate the risk score for data corresponding to entities within an outer cohort data subset based at least in part on an inner cohort definition. The risk score may be used by a computing device to perform one or more prediction-based actions. The predictive machine learning model may be applied to any prediction task that are concentrated on certain populations. Some examples, in the healthcare space, include but are not limited to, predicting future health outcomes using medical data and identifying members that are at higher risk of developing a diagnosis. In an example, the predictive machine learning model may predict medical diagnosis associated with members of a certain healthcare demographics and recommend procedures or treatment to a medical decision aid system.

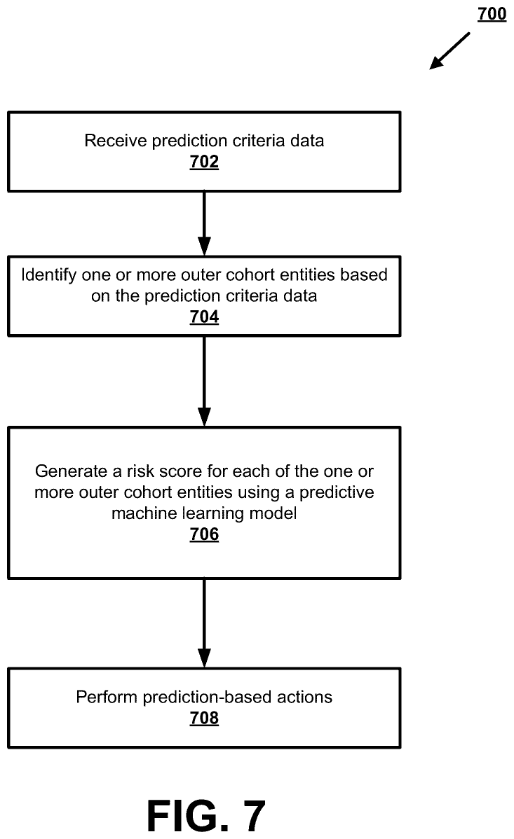

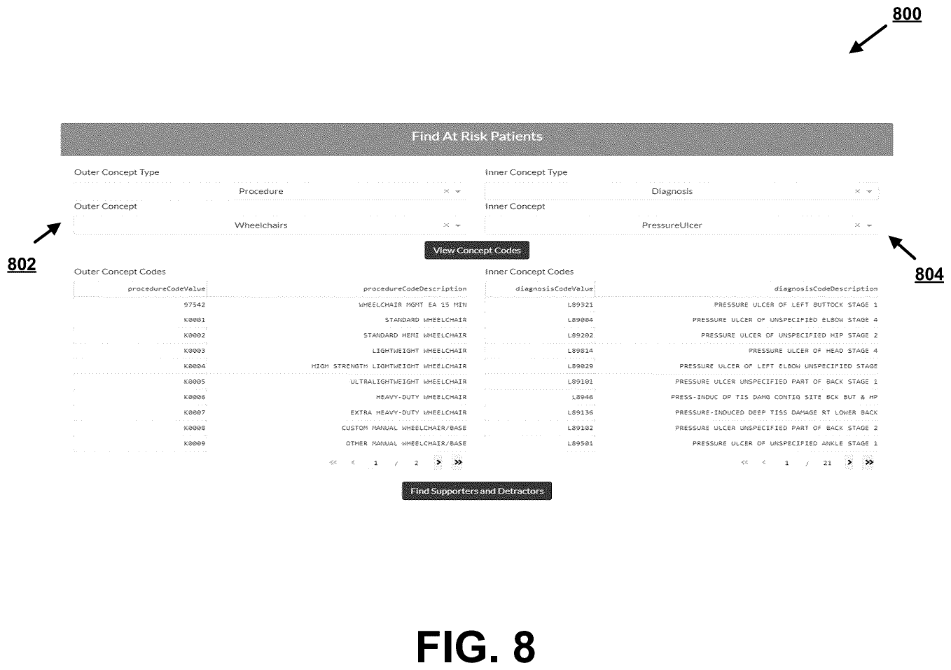

FIG. 7 is a flowchart diagram of an example process for performing predictive operations on a target data domain in accordance with some embodiments discussed herein. Via the various steps/operations of the process 700, the predictive data analysis computing entity 106 can use a predictive machine learning model to generate risk scores for predicting outcomes for a target data domain. The process 700 begins at step/operation 702 when the predictive data analysis computing entity 106 receives prediction criteria data. The prediction criteria data may be used to define a feature for prediction on a target data domain from a dataset. In some embodiments, the prediction criteria data may comprise outer cohort definition data and inner cohort definition data. As an example, an outer cohort data subset may be identified from the dataset based on the outer cohort definition data and one or more features for predictions may be determined based on the inner cohort definition data. Referring to FIG. 8, exemplary prediction criteria are presented for a prediction output user interface 800. As illustrated, prediction criteria comprise outer cohort definition data inputs 802 including outer concept type and outer concept, and inner cohort definition data inputs 804 including inner concept type and inner concept.

Referring to FIG. 7, at step/operation 704, the predictive data analysis computing entity 106, identifies one or more outer cohort entities based on the prediction criteria data. As an example, the one or more outer cohort entities may comprise an outer cohort subset including data associated with features that match the outer cohort definition data.

Referring back to FIG. 7, at step/operation 706, the predictive data analysis computing entity 106, using a predictive machine learning model, generates a risk score for each of the one or more outer cohort entities. In some embodiments, a risk score describes an output of the predictive machine learning model. A risk score may comprise a probability of an entity associated with features in an outer cohort data subset will become an entity associated with features in an inner cohort data subset corresponding to the inner cohort definition data. For example, the risk score may define a probability of wheelchair-bound patients (outer cohort data subset) that develop a pressure ulcer (inner cohort data subset). Other examples may include predicting cancer patients that develop adverse reactions to chemotherapy, and diabetics that develop foot problems.

At step/operation 708, the predictive data analysis computing entity 106 performs one or more prediction-based actions based at least in part on the risk scores generated by the predictive machine learning model. In some embodiments, performing the one or more prediction-based actions based at least in part on the risk scores includes displaying the one or more diagnosis or procedure recommendations using a prediction output user interface, such as the prediction output user interface 800 of FIG. 8. As depicted in FIG. 8, the prediction output user interface 800 displays an interface for identifying at risk patients based at least in part on concept codes associated with input of outer concept criteria and inner concept criteria.

In some embodiments, highest risk members (outer cohort entities) of any size group may be targeted by using an appropriate threshold of risk score. For example, a user can specify a confidence threshold in order to control the number of individuals the predictive machine learning model returns that have a high risk of developing specified conditions (e.g., based at least in part on inner cohort definition data). As such, a confidence threshold may be used to identify and prioritize members that are at higher risk of developing a given diagnosis. This allows healthcare providers and insurers to intervene with the patients who need care the most.

Accordingly, as described above, various embodiments of the present disclosure make important technical contributions to improving predictive accuracy of predictive machine learning models by identifying targeted cohort data subsets, which in turn improves training speed and training efficiency of training predictive machine learning models. It is well-understood in the relevant art that there is typically a tradeoff between predictive accuracy and training speed, such that it is trivial to improve training speed by reducing predictive accuracy, and thus the real challenge is to improve training speed without sacrificing predictive accuracy through innovative model architectures, see, e.g., Sun et al., Feature-Frequency-Adaptive On-line Trainingfor Fast and Accurate Natural Language Processing in 40(3) Computational Linguistic 563 at Abst. (“Typically, we need to make a tradeoff between speed and accuracy. It is trivial to improve the training speed via sacrificing accuracy or to improve the accuracy via sacrificing speed. Nevertheless, it is nontrivial to improve the training speed and the accuracy at the same time”). Accordingly, techniques that improve predictive accuracy without harming training speed, such as the techniques described herein, enable improving training speed given a constant predictive accuracy. In doing so, the techniques described herein improving efficiency and speed of training predictive machine learning models, thus reducing the number of computational operations needed and/or the amount of training data entries needed to train predictive machine learning models. Accordingly, the techniques described herein improve at least one of the computational efficiency, storage-wise efficiency, and speed of training machine learning models.

Accordingly, as described above, various embodiments of the present invention enable techniques for using feature data associated with one predictive task, such as a predictive task related to an inner cohort membership prediction, to train a predictive machine learning model that is configured to perform predictive inferences related to an outer cohort membership prediction. In this way, the noted embodiments of the present invention enable training a predictive machine learning model that is configured to perform predictive inferences related to an outer cohort membership prediction without having to store training data whose feature set is specifically generated for the outer cohort membership prediction task, which in turn reduces storage requirements for training the predictive machine learning model and thus enhances storage-wise efficiency of training the predictive machine learning model. Accordingly, various embodiments of the present invention improve storage-wise efficiency of training predictive machine learning models that are configured to perform outer cohort membership prediction tasks.

### VI. Conclusion

Many modifications and other embodiments will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. A computer-implemented method, in a data processing system comprising a processor and a memory, for performing risk prediction with respect to data subsets, the computer-implemented method comprising:
receiving, by a computing device, outer cohort definition data and inner cohort definition data, the outer cohort definition data representative of a target data domain with respect to a dataset, and the inner cohort definition data representative of a prediction feature with respect to the target data domain;
determining, by the computing device, one or more inner cohort features based at least in part on a knowledge graph data object using the inner cohort definition data, the knowledge graph data object including co-occurrence information of features from the dataset;
for each outer cohort entity of one or more outer cohort entities associated with features in an outer cohort data subset, generating, by the computing device and using a predictive machine learning model, a risk score representative of a propensity of the outer cohort entity being an inner cohort entity, wherein training the predictive machine learning model comprises:
identifying an outer cohort data subset from the dataset based at least in part on the outer cohort definition data;
for each entity associated with the outer cohort data subset, retrieving an input feature value set comprising input feature values associated with the outer cohort data entity that correspond to the one or more inner cohort features;
for each entity associated with the outer cohort data subset, determining a membership indicator to the inner cohort data subset based at least in part on using the knowledge graph data object to determine correlation values between the input feature values associated with the outer cohort data subset and the one or more inner cohort features;
generating training data based at least in part on: (i) each membership indicator, and (ii) each input feature value set; and
training the predictive machine learning model based at least in part on the training data; and
performing, by the computing device, one or more prediction-based actions based at least in part on the risk score.

2. The computer-implemented method of claim 1, wherein the dataset comprises a collection of data from one or more sources.

3. The computer-implemented method of claim 1, wherein the predictive machine learning model is based at least in part on at least one of neural network, random forest, logistic regression, and gradient boosting.

4. The computer-implemented method of claim 1, wherein the features associated with the dataset include one or more of: diagnosis codes, medication codes, demographics, and procedure codes.

5. The computer-implemented method of claim 1, wherein the correlation values comprise a ratio of odds of a condition being present for a given entity in the inner cohort data subset compared to another given entity not in the inner cohort data subset.

6. The computer-implemented method of claim 1, wherein the training data comprises, for each entity associated with the outer cohort data subset that is associated with an affirmative membership indicator, a training data entry that comprises the input feature value set for the entity and a ground-truth output that is generated based at least in part on the affirmative membership indicator.

7. An apparatus for performing risk prediction with respect to data subsets, the apparatus comprising at least one processor and at least one memory including program code, the at least one memory and the program code configured to, with the processor, cause the apparatus to at least:
receive outer cohort definition data and inner cohort definition data, the outer cohort definition data representative of a target data domain with respect to a dataset, and the inner cohort definition data representative of a prediction feature with respect to the target data domain;
determine one or more inner cohort features based at least in part on a knowledge graph data object using the inner cohort definition data, the knowledge graph data object including co-occurrence information of features from the dataset;
for each outer cohort entity of one or more outer cohort entities associated with features in an outer cohort data subset, generating, by the computing device and using a predictive machine learning model, a risk score representative of a propensity of the outer cohort entity being an inner cohort entity, wherein training the predictive machine learning model comprises:
identifying an outer cohort data subset from the dataset based at least in part on the outer cohort definition data;
for each entity associated with the outer cohort data subset, retrieving an input feature value set comprising input feature values associated with the outer cohort data entity that correspond to the one or more inner cohort features;
for each entity associated with the outer cohort data subset, determining a membership indicator to the inner cohort data subset based at least in part on using the knowledge graph data object to determine correlation values between the input feature values associated with the outer cohort data subset and the one or more inner cohort features;
generating training data based at least in part on: (i) each membership indicator, and (ii) each input feature value set; and
training the predictive machine learning model based at least in part on the training data; and
perform one or more prediction-based actions based at least in part on the risk score.

8. The apparatus of claim 7, wherein the dataset comprises a collection of data from one or more sources.

9. The apparatus of claim 7, wherein the predictive machine learning model is based at least in part on at least one of neural network, random forest, logistic regression, and gradient boosting.

10. The apparatus of claim 7, wherein the features associated with the dataset include one or more of: diagnosis codes, medication codes, demographics, and procedure codes.

11. The apparatus of claim 7, wherein the correlation values comprise a ratio of odds of a condition being present for a given entity in the inner cohort data subset compared to another given entity not in the inner cohort data subset.

12. The apparatus of claim 7, wherein the training data comprises, for each entity associated with the outer cohort data subset that is associated with an affirmative membership indicator, a training data entry that comprises the input feature value set for the entity and a ground-truth output that is generated based at least in part on the affirmative membership indicator.

13. A computer program product for performing risk prediction with respect to data subsets, the computer program product comprising at least one non-transitory computer-readable storage medium having computer-readable program code portions stored therein, the computer-readable program code portions configured to:
receive outer cohort definition data and inner cohort definition data, the outer cohort definition data representative of a target data domain with respect to a dataset, and the inner cohort definition data representative of a prediction feature with respect to the target data domain;
determine one or more inner cohort features based at least in part on a knowledge graph data object using the inner cohort definition data, the knowledge graph data object including co-occurrence information of features from the dataset;
for each outer cohort entity of one or more outer cohort entities associated with features in an outer cohort data subset, generating, by the computing device and using a predictive machine learning model, a risk score representative of a propensity of the outer cohort entity being an inner cohort entity, wherein training the predictive machine learning model comprises:
identifying an outer cohort data subset from the dataset based at least in part on the outer cohort definition data;
for each entity associated with the outer cohort data subset, retrieving an input feature value set comprising input feature values associated with the outer cohort data entity that correspond to the one or more inner cohort features;
for each entity associated with the outer cohort data subset, determining a membership indicator to the inner cohort data subset based at least in part on using the knowledge graph data object to determine correlation values between the input feature values associated with the outer cohort data subset and the one or more inner cohort features;
generating training data based at least in part on: (i) each membership indicator, and (ii) each input feature value set; and
training the predictive machine learning model based at least in part on the training data; and
perform one or more prediction-based actions based at least in part on the risk score.

14. The computer program product of claim 13, wherein the dataset comprises a collection of data from one or more sources.

15. The computer program product of claim 13, wherein the predictive machine learning model is based at least in part on at least one of neural network, random forest, logistic regression, and gradient boosting.

16. The computer program product of claim 13, wherein the features associated with the dataset include one or more of: diagnosis codes, medication codes, demographics, and procedure codes.

17. The computer program product of claim 13, wherein the correlation values comprise a ratio of odds of a condition being present for a given entity in the inner cohort data subset compared to another given entity not in the inner cohort data subset.

18. The computer program product of claim 13, wherein the training data comprises, for each entity associated with the outer cohort data subset that is associated with an affirmative membership indicator, a training data entry that comprises the input feature value set for the entity and a ground-truth output that is generated based at least in part on the affirmative membership indicator.
